(19) United States
(12) Patent Application Publication
(10) Pub. No.: US 2024/0407983 A1
(43) Pub. Date: Dec. 12, 2024

(54) (R,E)-3,7-DIMETHYLNON-6-ENAL, ISOMERIC MIXTURES OF THE COMPOUND AND THE USE IN PERFUMERY

(71) Applicant: GIVAUDAN SA, VERNIER (CH)

(72) Inventor: Marc LINIGER, Kemptthal (CH)

(21) Appl. No.: 18/698,849

(22) PCT Filed: Oct. 4, 2022
(86) PCT No.: PCT/EP2022/077520

§ 371 Date: Apr. 5, 2024

(30) Foreign Application Priority Data
Oct. 5, 2021 (GB) 2114238.5

(51) Int. Cl. A61K 8/33; A61K 8/34; A61Q 5/02; A61Q 13/00; A61Q 19/10; C07C 33/025; C07C 33/05; C07C 47/21

(52) U.S. Cl. CPC A61K 8/33; A61K 8/342; A61Q 5/02; A61Q 13/00; A61Q 19/10; C07C 33/025; C07C 33/05; C07C 47/21

(57) ABSTRACT

It is provided compound of formula (R,E)-(Ia), which is (R,E)-3,7-dimethylnon-6-enal, isomeric mixtures of the compound and the use in perfumery.

### TECHNICAL FIELD

The present invention relates generally to a novel organic compound and its use as fragrance ingredient. The invention also relates to fragrance compositions and to consumer products comprising the compound. It further relates to a method of improving, enhancing or modifying a consumer product base by addition of the compound.

### BACKGROUND

Citral (3,7-dimethylocta-2,6-dienal) and Citronellal (3,7-dimethyloct-6-enal) are known compounds with a citrus, lemon like odor. Both are used in perfumery, however, Citral is known to be an allergen. Overall, citrus notes are complex, and can have many different aspects.

It is therefore desirable to provide new or improved fragrance compounds with citrus and optionally other notes.

### SUMMARY

In accordance with a first aspect of the present invention there is provided a novel compound with citrus and floral odor notes.

In accordance with a second aspect of the present invention there is provided an isomeric mixture comprising the novel compound.

In accordance with a third aspect of the present invention there is provided a use of the novel compound or the isomeric mixture as fragrance ingredient.

In accordance with a fourth aspect of the present invention there are provided fragrance compositions and consumer products comprising the novel compound or the isomeric mixture. Furthermore, the fragrance compositions and consumer products may comprise certain alcohols.

In accordance with a fifth aspect of the present invention there is provided a method of improving, enhancing or modifying a consumer product base by means of addition of the novel compound to the base.

Certain embodiments of any aspect of the present invention may provide one or more of the following advantages:
provision of novel fragrance compound with citrus and floral odor notes,
provision of a compound without allergenic properties,
provision of a highly performing compound with low odor threshold.

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### DETAILED DESCRIPTION

The present invention is based on the surprising finding that a homologue of Citronellal formally extended by one methyl group is a fragrance compound with a significantly different odor description, and a remarkably enhanced performance.

In a first aspect of the invention, there is provided a compound of formula (R,E)-(Ia), which is (R,E)-3,7-dimethylnon-6-enal

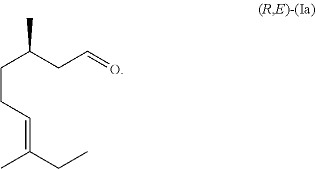

Said compound is a useful fragrance ingredient surprisingly possessing citrus and floral odor notes. In addition, it has a remarkably low Gas Chromatography (GC) threshold of 0.013 ng, in comparison to the GC threshold of Citral of 4.0 ng or Citronellal of 2.2 ng. Such a performant molecule, with a GC threshold by a factor 100 lower than the related structures, can be used in lower dosage, thereby reducing the needed amount of the compound. The perfumistic benefit of the compound of the invention depends on the stereochemistry: only one stereoisomer out of four is a strong odorant, while the others show only very weak odorant properties.

For example, compound (R,E)-(Ia) can be employed at least partially as replacer for Citral, to at least lower the required amounts of Citral, thereby reducing coloration issues associated with Citral, and minimizing the amount of the allergenic compound.

3,7-dimethylnon-6-enal has been reported previously. For example, a method of its preparation is disclosed in DE2050677A1. However, its fragrance properties have not been described.

The compound (R,E)-(Ia) of the present invention can be used as pure stereoisomer, or as mixture with one or more of its stereoisomers, for examples in groups of diasteroisomers. In contrast to the compound of the present invention, the other stereoisomers have much weaker odor properties.

(S,E)-3,7-dimethylnon-6-enal ((S,E)-(Ia)) has a GC threshold of 1.4 ng, and a waxy, fatty odor with citrus and floral rosy notes. (R,Z)-3,7-dimethylnon-6-enal ((R,Z)-(Ia)) has a GC threshold of 1.2 ng, and its odor is described as waxy citrus, fruity. (S,Z)-3,7-dimethylnon-6-enal ((S,Z)-(Ia)) has a GC threshold of 0.75 ng, and a waxy, fatty, plastic odor with citrus notes.

As the other isomers do not possess a significant odor, they can be tolerated as admixtures to the compound of the present invention. By the use of such an admixture, the costs of isomer separation can be avoided, making the overall production costs lower. Resolving stereoisomers adds to the complexity of manufacture and purification of the compound, and so it is preferred to use the compound as mixture of its stereoisomers simply for economic reasons. However, if it is desired to prepare individual stereoisomers, this may be achieved according to methods known in the art, e.g. stereoselective synthesis, preparative HPLC and GC.

So in a further aspect of the invention, there is provided an isomeric mixture (referred to in the following text as the isomeric mixture) comprising (R,E)-3,7-dimethylnon-6-enal ((R,E)-(Ia)) and at least one other compound selected from the group consisting of (S,E)-3,7-dimethylnon-6-enal ((S,E)-(Ia)), (R,Z)-3,7-dimethylnon-6-enal ((R,Z)-(Ia)), and (S,Z)-3,7-dimethylnon-6-enal ((S,Z)-(Ia)).

In another aspect of the invention, the compound of formula (R,E)-(Ia) is provided in highly enriched or essentially pure form, with low amounts or essentially free of compounds (S,E)-(Ia), (R,Z)-(Ia), and/or (S,Z)-(Ia). That means that the compound of formula (R,E)-(Ia) is present in at least 90 weight %, particularly in at least 95 weight %, more particularly in at least 98 weight %, even more particularly in 99 weight % or higher.

In another aspect of the invention, the isomeric mixture is provided, comprising a considerable amount of the compound of formula (R,E)-(Ia) along with compounds (S,E)-(Ia), (R,Z)-(Ia), and/or (S,Z)-(Ia). That means that the compound of formula (R,E)-(Ia) is present in at least 20 weight %, particularly in at least 30 weight %, more particularly in at least 40 weight %, even more particularly in 50 weight % or higher. The weight ratio of the compound of formula (R,E)-(Ia) to the sum of the compounds of formula (S,E)-(Ia), (R,Z)-(Ia), and/or (S,Z)-(Ia) in the isomeric mixture may range from about 2:8 to about 99:1. For example, the isomeric mixture may comprise compounds (R,E)-(Ia), (S,E)-(Ia), (R,Z)-(Ia), and (S,Z)-(Ia) in a ratio of 1:1:1:1.

In another aspect of the invention, a mixture of (R)-isomers (R,E)-(Ia) and (R,Z)-(Ia) is provided, in pure or enantio-enriched form.

In another aspect of the invention, a mixture of (E)-isomers (R,E)-(Ia) and (S,E)-(Ia) is provided, in pure or double bond isomer enriched form.

In a further aspect of the invention, there is provided a use of the compound of formula (R,E)-(Ia) or of the isomeric mixture comprising (R,E)-(Ia) and at least one other compound selected from the group consisting of (S,E)-(Ia), (R,Z)-(Ia), and/or (S,Z)-(Ia), as fragrance ingredient.

In another aspect of the present invention, there is provided a perfume composition comprising the compound of formula (R,E)-(Ia) or the isomeric mixture comprising (R,E)-(Ia) and at least one other compound selected from the group consisting of (S,E)-(Ia), (R,Z)-(Ia), and/or (S,Z)-(Ia).

The compound or the isomeric mixture of the invention may be used alone, or in combination with known odorant molecules selected from the extensive range of natural products, and synthetic molecules currently available, such as essential oils, alcohols, aldehydes and ketones, ethers and acetals, esters and lactones, macrocycles and heterocycles, and/or in admixture with one or more ingredients or excipients conventionally used in conjunction with odorants in perfume compositions, for example, carrier materials, and other auxiliary agents commonly used in the art.

As used herein, “carrier material” means a material which is practically neutral from a odorant point of view, i.e. a material that does not significantly alter the organoleptic properties of odorants.

The term “auxiliary agent” refers to ingredients that might be employed in a perfume composition for reasons not specifically related to the olfactive performance of said composition. For example, an auxiliary agent may be an ingredient that acts as an aid to processing a fragrance ingredient or ingredients, or a composition containing said ingredient(s), or it may improve handling or storage of a fragrance ingredient or composition containing same. It might also be an ingredient that provides additional benefits such as imparting color or texture. It might also be an ingredient that imparts light resistance or chemical stability to one or more ingredients contained in a perfume composition. A detailed description of the nature and type of adjuvants commonly used in perfume compositions containing same cannot be exhaustive, but it has to be mentioned that said ingredients are well known to a person skilled in the art.

As used herein, “perfume composition” means any composition comprising the compound according to formula (R,E)-(la) or the isomeric mixture and a base material, e.g. a diluent conventionally used in conjunction with odorants, such as diethyl phthalate (DEP), dipropylene glycol (DPG), isopropyl myristate (IPM), pentane-1,2-diol, triethyl citrate (TEC) and alcohol (e.g. ethanol). Optionally, the composition may comprise an anti-oxidant adjuvant. Said anti-oxidant may be selected from Tinogard® TT (BASF), Tinogard® Q (BASF), Tocopherol (including its isomers, CAS 59-02-9; 364-49-8; 18920-62-2; 121854-78-2), 2,6-bis(1, 1-dimethylethyl)-4-methylphenol (BHT, CAS 128-37-0) and related phenols, hydroquinones (CAS 121-31-9).

The following list comprises examples of known odorant molecules, which may be combined with the compound according to formula (R,E)-(Ia) or the isomeric mixture:
Essential oils and extracts, e.g. castoreum, costus root oil, oak moss absolute, geranium oil, tree moss absolute, basil oil, fruit oils, such as bergamot oil and mandarine oil, myrtle oil, palmarose oil, patchouli oil, petitgrain oil, jasmine oil, rose oil, sandalwood oil, wormwood oil, lavender oil and/or ylang-ylang oil;
Alcohols, e.g. cinnamic alcohol ((E)-3-phenylprop-2-en-1-ol); cis-3-hexenol ((Z)-hex-3-en-1-ol); Citronellol (3,7-dimethyloct-6-en-1-ol); dihydro myrcenol (2,6-dimethyloct-7-en-2-ol); Ebanol™ ((E)-3-methyl-5-(2,2,3-trimethylcyclopent-3-en-1-yl)pent-4-en-2-ol); eugenol (4-allyl-2-methoxyphenol); ethyl linalool ((E)-3,7-dimethylnona-1,6-dien-3-ol); farnesol ((2E,6Z)-3,7,11-trimethyldodeca-2,6,10-trien-1-ol); geraniol ((E)-3,7-dimethylocta-2,6-dien-1-ol); Super Muguet™ ((E)-6-ethyl-3-methyloct-6-en-1-ol); linalool (3,7-dimethylocta-1,6-dien-3-ol); menthol (2-isopropyl-5-methylcyclohexanol); Nerol (3,7-dimethyl-2,6-octadien-1-ol); phenyl ethyl alcohol (2-phenylethanol); Rhodinol™ (3,7-dimethyloct-6-en-1-ol); Sandalore™ (3-methyl-5-(2,2,3-trimethylcyclopent-3-en-1-yl) pentan-2-ol); terpineol (2-(4-methylcyclohex-3-en-1-yl)propan-2-ol); or Timberol™ (1-(2,2,6-trimethylcyclohexyl)hexan-3-ol); 2,4,7-trimethylocta-2,6-dien-1-ol, and/or [1-methyl-2(5-methylhex-4-en-2-yl)cyclopropyl]-methanol;
Aldehydes and ketones, e.g. anisaldehyde (4-methoxybenzaldehyde); alpha amyl cinnamic aldehyde (2-benzylideneheptanal); Georgywood™ (1-(1,2,8,8-tetramethyl-1,2,3,4,5,6,7,8-octahydronaphthalen-2-yl)ethanone); Hydroxycitronellal (7-hydroxy-3,7-dimethyloctanal); Iso E Super® (1-(2,3,8,8-tetramethyl-1,2,3,4,5,6,7,8-octahydronaphthalen-2-yl)ethanone); Isoraldeine® ((E)-3-methyl-4-(2,6,6-trimethylcyclohex-2-en-1-yl)but-3-en-2-one); Hedione® (methyl 3-oxo-2-pentylcyclopentaneacetate); Nympheal (3-(4-isobutyl-2-methylphenyl)propanal); Mahonial (5,9-dimethyl-9-hydroxy-decen-4-al); maltol; methyl cedryl ketone; methylionone; verbenone; and/or vanillin;
Ether and acetals, e.g. Ambrox® (3a,6,6,9a-tetramethyl-2,4,5,5a,7,8,9,9b-octahydro-1H-benzo[e][1]benzofuran); geranyl methyl ether ((2E)-1-methoxy-3,7-dimethylocta-2,6-diene); rose oxide (4-methyl-2-(2-methylprop-1-en-1-yl)tetrahydro-2H-pyran); and/or Spirambrene® (2′,2′,3,7,7-pentamethylspiro[bicyclo[4.1.0]heptane-2,5′-[1,3]dioxane]);
Esters and lactones, e.g. benzyl acetate; cedryl acetate ((1S,6R,8aR)-1,4,4,6-tetramethyloctahydro-1H-5,8a-methanoazulen-6-yl acetate); γ-decalactone (6-pentyltetrahydro-2H-pyran-2-one); Helvetolide® (2-(1-(3,3-dimethylcyclohexyl)ethoxy)-2-methylpropyl propionate); γ-undecalactone (5-heptyloxolan-2-one); and/or vetiveryl acetate ((4,8-dimethyl-2-propan-2-ylidene-3,3a,4,5,6,8a-hexahydro-1H-azulen-6-yl) acetate
Macrocycles, e.g. Ambrettolide ((Z)-oxacycloheptadec-10-en-2-one); ethylene brassylate (1,4-dioxacycloheptadecane-5,17-dione); and/or Exaltolide® (16-oxacyclohexadecan-1-one); and
Heterocycles, e.g. isobutylquinoline (2-isobutylquinoline).

Furthermore, there is provided a perfume composition comprising the compound of formula (R,E)-(Ia) or the isomeric mixture comprising (R,E)-(Ia) and at least one other compound selected from the group consisting of (S,E)-(Ia), (R,Z)-(Ia), and/or (S,Z)-(Ia), and at least one or more compounds selected from the group consisting of a compound of formula (Ib), (Ic) and (II)

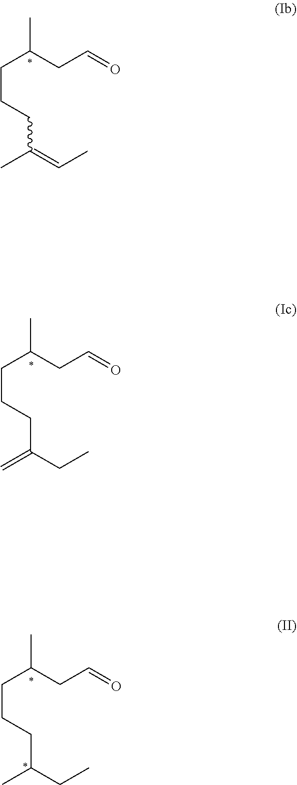

wherein the wavy bond is indicating an unspecified configuration of the adjacent double bond, and the star is indicating a stereocenter at the C-atom.

The compounds of formula (Ib), (Ic) and (II) do have at least one stereocenter at a C atom or at least one double bond. Therefore, the compounds can exist in up to four isomers, varying in the configuration of the stereocenter and/or double bond. The compounds as herein described occur in any of the possible isomer, or as a mixture of some or all possible isomers.

The compounds of formula (Ib) and (Ic) do not have a strong odor. Therefore, they can be accepted in a mixture with compound (R,E)-(Ia), if they arise as by-products in the production of compound (R,E)-(Ia), and if their separation is adding costs.

The compound of formula (II) possess a fatty, waxy, floral, rosy citrus odor, and is also very performing (GCTH 0.046 ng). In mixture with compound (R,E)-(Ia) a powerful blend with a broader olfactive facets can be provided. For example, a perfume composition comprising the compound of formula (R,E)-(Ia) or of the isomeric mixture comprising (R,E)-(Ia) and the compound of formula (II) in up to 3% by weight is liked by perfumers.

In another aspect of the present invention, there is provided a perfume composition comprising the compound of formula (R,E)-(Ia), or the isomeric mixture comprising (R,E)-(Ia) and at least one other compound selected from the group consisting of (S,E)-(Ia), (R,Z)-(Ia), and/or (S,Z)-(Ia), and at least one compound of formula (III)

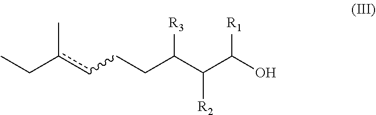

wherein
[symbol] is indicating a carbon-carbon single or double bond,
the wavy bond is indicating an unspecified configuration of the adjacent double bond, if present,
and R₁, R₂ and R₃ are independently selected from H or Me,
or wherein R₁ is H or Me, and R₂ and R₃ are forming together with the carbon atoms they are attached to a cyclopropyl ring.

The compounds of formula (III) can have up to three stereocenters at a C atom and/or at least one double bond. Therefore, the compounds can exist in up to sixteen isomers, varying in the configuration of the stereocenter and/or double bond. The compounds as herein described occur in any of the possible isomer, or as a mixture of some or all possible isomers.

The compounds of formula (III) are alcohol derivatives of compound (Ia) and possess floral notes and low odor thresholds. The compounds of formula (III) in admixture with compound (R,E)-(Ia) provide novel floral accords.

Compound Ia refers to an isomer with unspecified configuration, or to a mixture of two or more isomers described above.

For example, the above mentioned fragrance composition further comprises at least one compound of formula (III), which is selected from the group consisting of a compound of formula (IIIa), (IIIb), (IIIc), and (IIId)

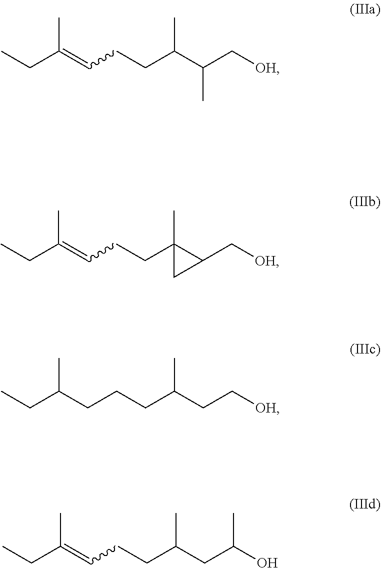

wherein the wavy bond is indicating an unspecified configuration of the adjacent double bond, if present.

Furthermore, there is provided a perfume composition comprising the compound of formula (R,E)-(Ia), or of the isomeric mixture comprising (R,E)-(Ia) and at least one other compound selected from the group consisting of (S,E)-(Ia), (R,Z)-(Ia), and/or (S,Z)-(Ia), and at least one or more compounds selected from the group consisting of a compound of formula (Ib), (Ic), (II), and (III).

While some of the compounds of formula (III) are known, some have not been reported before, and are therefore novel. So in another aspect of the present invention, there is provided a compound of formula (III)

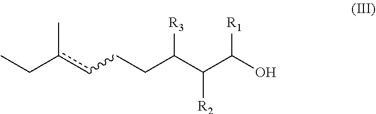

wherein
[symbol] is indicating a carbon-carbon single or double bond,
the wavy bond is indicating an unspecified configuration of the adjacent double bond, if present,
and R₁, R₂ and R₃ are independently selected from H or Me,
or wherein R₁ is H or Me, and R₂ and R₃ are forming together with the carbon atoms they are attached to a cyclopropyl ring,
with the proviso that the compound is not 3,7-dimethylnonan-1-ol and 3,7-dimethylnon-6-en-1-ol.

For example, the compound of formula (III) can be selected from the group consisting of a compound of formula (IIIa), (IIIb), and (IIId)

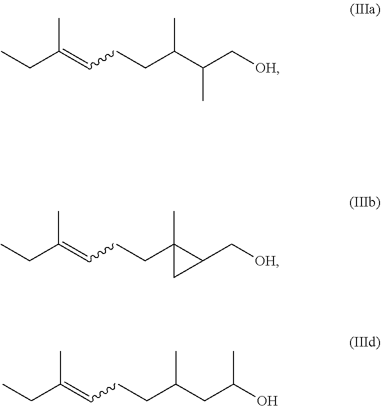

wherein the wavy bond is indicating an unspecified configuration of the adjacent double bond.

The compound of formula (IIIa) (2,3,7-trimethylnon-6-en-1-ol) has a floral rosy odor and a GC threshold of 0.04 ng. The compound of formula (IIIb) ((2-methyl-2-(4-methylhex-3-en-1-yl)cyclopropyl)methanol) has a floral rosy fruity litchy odor and a GC threshold of 1.2 ng. And the compound of formula (IIId) (4,8-dimethyldec-7-en-2-ol) has a floral pomelol green odor, but a relatively high GC threshold of 15 ng.

In another aspect of the present invention, there is provided a fragrance composition comprising at least one or more compounds of formula (III).

In another aspect of the present invention, there is provided a perfume composition comprising the compound of formula (R,E)-(Ia), or the isomeric mixture comprising (R,E)-(Ia) and at least one other compound selected from the group consisting of (S,E)-(Ia), (R,Z)-(Ia), and/or (S,Z)-(Ia), and at least one compound of formula (X)

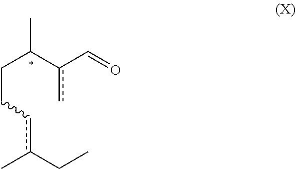

wherein
[symbol] is indicating a carbon-carbon single or double bond,
the wavy bond is indicating an unspecified configuration of the adjacent double bond, and the star is indicating a stereocenter at the C-atom.

The compounds of formula (X) in admixture with compound (R,E)-(Ia) provide novel citrus accords.

The compounds of formula (X) been used as intermediates in preparation of the compounds of formula (III) and were also found to have interesting fragrance properties. So in another aspect of the present invention, there is provided a compound of formula (X)

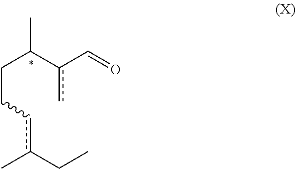

wherein
[symbol] is indicating a carbon-carbon single or double bond,
the wavy bond is indicating an unspecified configuration of the adjacent double bond, and
the star is indicating a stereocenter at the C-atom.

For example, the compound of formula (X) can be selected from the group consisting of 3,7-dimethyl-2-methylenenon-6-enal and 2,3,7-trimethylnon-6-enal.

In yet another aspect of the present invention, there is provided a consumer product comprising the compound of formula (R,E)-(Ia), or the isomeric mixture or the fragrance composition as defined above, and a consumer product base.

The consumer product for example is selected from fine fragrance, personal care products (body care products, cosmetic products) fabric care products, home care products and air care products. As used herein, “consumer product base” means a composition for use as a consumer product to fulfill specific actions, such as cleaning, softening, and caring or the like.

Personal care products to which the compound of formula (R,E)-(Ia) or mixtures comprising it can be added include for example all kinds of body care products. Especially interesting products are hair care products, for example shampoos, conditioners and hairsprays, and skin care products, like lotions or creams. Furthermore, the compound of formula (R,E)-(Ia) or mixtures comprising it may be added to soaps, bath and shower gels and deodorants. Also the compound of (R,E)-(Ia) or mixtures comprising it can be added to cosmetic products.

Home care products to which the compound of formula (R,E)-(Ia) or mixtures comprising it can be added include all kinds of detergents, window cleaners, hard surface cleaners, all-purpose cleaners and furniture polishes. Preferably, the products are liquids, e.g. fabric detergent or conditioner compositions.

The compound according to formula (R,E)-(Ia) or the isomeric mixture comprising it may be used in a broad range of perfumed consumer products, e.g. in any field of fine and functional perfumery, such as perfumes, air care products, household products, laundry products, body care products and cosmetics. The compound can be employed in widely varying amounts, depending upon the specific article and on the nature and quantity of other odorant ingredients. The proportion is typically from 0.0001 to 5 weight % of the article. In one embodiment, the compound of the present invention or the isomeric mixture comprising (R,E)-(Ia) may be employed in a fabric softener in an amount from 0.001 to 0.3 weight % (e.g. 0.01 to 0.1 including 0.05 weight %). In another embodiment, the compound (R,E)-(Ia) or the isomeric mixture comprising (R,E)-(Ia) may be used in fine perfumery but also in consumer products like shampoo, fabric softener or fabric detergents, in amounts from 0.001 to 30 weight % (e.g. up to about 10 or up to 20 weight %), more preferably between 0.01 and 5 weight %. However, these values are given only by way of example, since the experienced perfumer may also achieve effects or may create novel accords with lower or higher concentrations.

In one embodiment there is provided a consumer product comprising an acceptable amount of the compound (R,E)-(Ia) or the isomeric mixture comprising (R,E)-(Ia). For example, the fragranced article may comprise 0.000001 weight % to 90 weight % (including 0.00001 weight %; 0.0001 weight %, 0.001 weight %, 0.01 weight %, 0.05 weight %, 0.1 weight %, 0.5 weight %, 1 weight %, 5 weight %, 8 weight %, 10 weight %, 15 weight %, 20 weight %, 25 weight %, 30weight %, 50 weight %, 60 weight %, 65 weight %) of the compound or isomeric mixture based on the total amount of the article.

The compound (R,E)-(Ia) or the isomeric mixture comprising it may be employed in a consumer product base simply by directly mixing the compound of the present invention, or a perfume composition comprising the compound (R,E)-(Ia) or the isomeric mixture comprising (R,E)-(Ia), or a mixture thereof, with the consumer product base, or it may, in an earlier step, be entrapped with an entrapment material, for example, polymers, capsules, microcapsules and nanocapsules, liposomes, film formers, absorbents such as carbon or zeolites, cyclic oligosaccharides and mixtures thereof, and then mixed with the consumer product base.

Thus, in a further aspect, the invention additionally provides a method of manufacturing a consumer product, comprising the incorporation of the compound (R,E)-(Ia) or the isomeric mixture comprising (R,E)-(Ia), as a fragrance ingredient, either by directly admixing the compound to the consumer product base or by admixing a perfume composition comprising the compound (R,E)-(Ia) or the isomeric mixture comprising (R,E)-(Ia), which may then be mixed with a consumer product base, using conventional techniques and methods. Through the addition of an olfactory acceptable amount of the compound of the present invention as hereinabove described the odor notes of a consumer product base will be improved, conferred, enhanced, or modified.

Thus, the invention furthermore provides in another aspect a method of improving, conferring, enhancing or modifying a consumer product base by means of addition thereto of an olfactory acceptable amount of compound (R,E)-(Ia), or the isomeric mixture or the fragrance composition comprising compound (R,E)-(la).

In a further aspect, the invention furthermore provides a method of improving, conferring, enhancing or modifying a consumer product base by means of addition thereto of an olfactory acceptable amount of the compound of formula (III) or the fragrance composition comprising the compound of formula (III).

The compound of formula (R,E)-(Ia), and the other compounds can be prepared in different processes, for example by oxidation of the corresponding primary alcohol (IV).

Said corresponding primary alcohol can be obtained in a two-step process from ethyl linalool (V) via ethyl geraniol/ethyl nerol (VI):

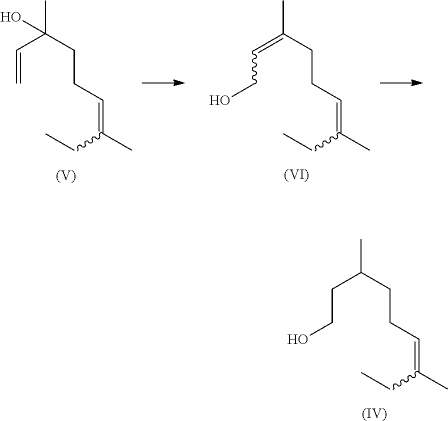

The primary alcohol can either be obtained in the form of individual isomers by stereoselective synthesis, or as a mixture of isomers that is further separated.

For example, the mixture of the alcohol isomers (IV) can be transformed to the corresponding 3,7-dimethylnon-6-en-1-yl 4-nitrobenzoate (VII) to enable for separation into individual isomers by SFC (supercritical fluid chromatography), followed by ester hydrolysis to the individual compounds (R,E)-(IV), (S,E)-(IV), (R,Z)-(IV), and (S,Z)-(IV).

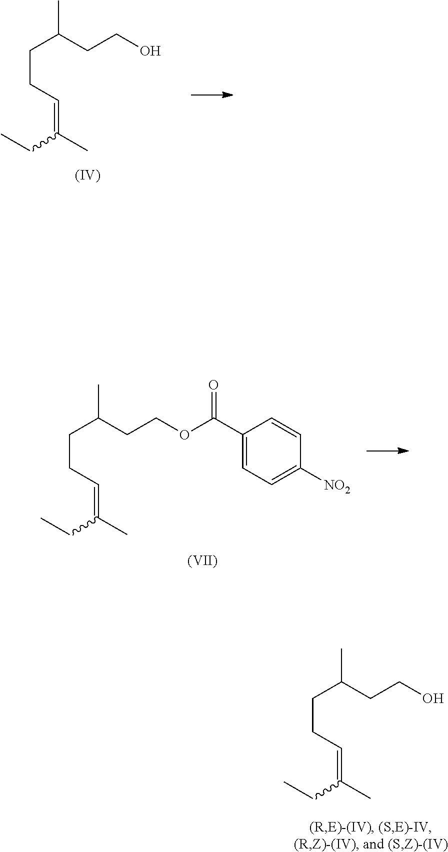

In one embodiment, the starting material for the process to prepare the compound of formula (R,E)-(Ia) is obtained from renewable resources. With such a starting material also the final product is accessible from renewable resources.

In a further aspect, the compounds of the present invention are biodegradable, as demonstrated by the Manometric Respirometry test (OECD guideline for the testing of materials No. 301F, Paris 1992).

The invention is now further described with reference to the following non-limiting examples. These examples are for the purpose of illustration only and it is understood that variations and modifications can be made by one skilled in the art.

### EXAMPLES

### General

All products were purified after work-up by either flash chromatography (FC) or distillation. Unless otherwise noted, a mixtures of heptane and methyl tert-butyl ether (MTBE) were used as eluent. ¹H and ¹³C NMR spectra were measured in C₆D₆ or CDCl₃. ¹H NMR spetra were referenced to the residual hydrogen signal of the deuterated solvent (¹H 7.27 ppm, ¹³C 77.0 ppm for CDCl₃; ¹H 7.16 ppm, ¹³C 128.4 ppm for C₆D₆) and are reported as follows: chemical shifts (δ ppm), coupling constants J in Hz. GC-MS analyses were run on a MSD5975mass spectrometer and are reported as m/z list (relative intensity). Electron ionization (EI) was run at 70 eV. Odor description refers to the odor of the isomeric mixture of the compounds unless otherwise indicated.

### Example 1: Synthesis of All Four Isomers of 3,7-dimethylnon-6-enal (R,E)-(Ia), (S,E)-(Ia), (R,Z)-(Ia), (S,Z)-(Ia)

### a) Preparation of 3,7-dimethylnon-6-en-1-ol (IV)

Ethyl Citronellol (IV) has been prepared in a two-step process, starting from ethyl linalool (V) via ethyl geraniol/ethyl nerol (VI):
i) 3,7-Dimethylnona-2,6-dien-1-ol (VI)

Under N₂, a 1000 mL 3-neck round bottomed flask equipped with a condenser was charged with ethyl linalool (V, 637.8 g, 3.79 mol), VO(OSiPh₃)₃ (20.1 g, 0.022 mol) and PhMe (139 g), and the solution was brought to reflux (heating plate set at 110° C.). The progress of the reaction was followed by GC until the equilibrium mixture of 70:30 ethyl linalool: ethyl geraniol/ethyl nerol (3,7-dimethylnona-2,6-dien-1-ol) was observed. After 3 h total reaction time, the reaction mixture was cooled to room temperature. PhMe was removed which afforded a crude light brown liquid (667 g). The reaction mixture was passed through a Wiped-film Evaporator (length=16 cm, OD 5.5 cm, 80° C., 0.04 mbar) which provided recovered VO(OSiPh₃)₃ (17 g) and crude material (ethyl linalool+ethyl geraniol/ethyl nerol mixture) (562.8 g). The crude material was filtered, and fractional distillation (Sulzer packed column (length=50 cm, ID 30 mm)) afforded ethyl linalool (250.1 g) and then ethyl geraniol/ethyl nerol (VI, 92 g, 100% purity, 48% isolated yield) as a mixture of four isomers in a 1:3.36:3.51:5.59 ratio.

Mixture of E/E, E/Z, Z/E and Z/Z isomers:

¹H-NMR (CDCl₃, 400 MHZ): δ=5.47-5.37 (m, 4×1H), 5.14-5.03 (m, 4×1H), 4.14 (d, J=6.8 Hz, 2×2H), 4.11-4.07 (m, 2×2H), 2.16-1.94 (m, 4×6H), 1.89 (br s, 4×1H), 1.75 (m, 2×3H), 1.67 (m, 4×3 H), 1.60 (s, 2×3H), 1.00-0.94 (m, 4×3H) ppm.

¹³C-NMR (CDCl₃, 100 MHZ): δ=139.6 (s), 139.5 (s), 139.34 (s), 139.28 (s), 137.9 (s), 137.7 (s), 137.3 (s), 137.1 (s), 124.43 (d), 124.37 (d), 123.42 (d), 123.35 (d), 122.3 (d), 122.1 (d), 59.1 (t), 58.78 (t), 58.76 (t), 39.8 (t), 39.5 (t), 32.22 (t), 32.19 (t), 31.9 (t), 26.3 (t), 26.2 (t), 26.1(t), 25.9 (t), 24.7 (t), 24.6 (t), 23.3 (q), 22.72 (q), 22.68 (q), 16.1 (q), 15.8 (q), 12.73 (q), 12.69(q), 12.67 (q), 12.6 (q) ppm.

Major E/E isomer:

¹³C-NMR (CDCl₃, 100 MHZ): δ=139.3 (s), 137.1 (s), 123.4 (d), 122.3 (d), 59.1 (t), 39.5 (t), 32.2 (t), 26.2 (t), 16.1 (q), 15.8 (q), 12.7 (q) ppm.

Peak 1. MS (El, Rt 6.43 min., 70 eV): 168 (1, [M]+⋅), 121 (9), 93 (17), 84 (14), 83 (33), 67(15), 55 (100), 53 (10), 41 (39), 39 (15), 29 (11).

Peak 2. MS (El, Rt 6.51 min., 70 eV): 168 (1, [M]+⋅), 121 (9), 93 (18), 84 (14), 83 (35), 67(15), 55 (100), 53 (10), 41 (38), 39 (14), 29 (10).

Peak 3. MS (El, Rt 6.62 min., 70 eV): 168 (0, [M]+⋅), 137 (9), 93 (9), 84 (8), 83 (37), 67 (13), 55 (100), 53 (9), 41 (36), 39 (12), 29 (10).

Peak 4. MS (El, Rt 6.67 min., 70 eV): 168 (1, [M]+⋅), 93 (9), 84 (8), 83 (39), 67 (13), 55 (100), 53 (9), 43 (8), 41 (35), 39 (12), 29 (9).
ii) 3,7-Dimethylnon-6-en-1-ol (IV)

Under air atmosphere, 3,7-dimethylnona-2,6-dien-1-ol (VI, 92 g, 0.547 mol, 100% purity) and MeOH (108 g) were added to a 500 mL autoclave vessel, followed by Raney Nickel (0.92 g, added in one shot in pellet form), and the vessel was sealed. Whilst stirring (600 rpm), the autoclave was flushed thrice with N₂ (1.8 bar), then three times with H₂ (2.0 bar). After adjusting the H₂-pressure to 10 bar, the autoclave was heated to 50° C. while stirring was increased to 1500 rpm. The reaction progress was followed by sampling. At t=6 h 39 min the heating of the autoclave and the H₂-flow were stopped. Once cooled, the pressure was released, and the autoclave was flushed three times with N₂ (1.8 bar). The reaction mixture was filtered to remove the heterogeneous catalyst, and MeOH was removed which provided 3,7-dimethylnon-6-en-1-ol (II, 78.6 g, 86.9% purity, 79.8% yield) as a colorless liquid, comprising two double bond isomers in a 2:1 ratio.

¹H-NMR (CDCl₃, 400 MHZ): δ=5.14-5.03 (m, 2×1H, E+Z), 3.74-3.60 (m, 2×2H, E+Z), 2.09-1.89 (m, 2×4H, E+Z), 1.67 (q, J=1.2 Hz, 3H, Z), 1.60 (sb, 3H, E), 1.66-1.13 (m, 2×5H, E+Z), 0.98 (t, J=7.5 Hz, 3H, E), 0.96 (t, J=7.5 Hz, 3H, Z), 0.91 (d, J=6.7 Hz, 3H, E), 0.91 (d, J=6.6 Hz, 3H, Z) ppm.

¹³C-NMR (CDCl₃, 100 MHZ): δ=137.0 (s, Z), 136.7 (s, E), 124.3 (d, Z), 123.1 (d, E), 61.1 (2t, E+Z), 39.8 (2t, E+Z), 37.5 (t, Z), 37.2 (t, E), 32.3 (t, E), 29.2 (2d, E+Z), 25.3 (t, E), 25.0 (t, Z), 24.7 (t, Z), 22.8 (q, Z), 19.5 (2q, E+Z), 15.8 (q, E), 12.8 (q, Z), 12.7 (q, E) ppm.

Peak 1. MS (El, Rt=6.43 min., 70 eV): 170 (3, [M]+⋅), 123 (39), 95 (18), 83 (18), 81 (42), 71 (18), 70 (19), 69 (21), 67 (26), 55 (100), 41 (45).

Peak 2. MS (El, Rt=6.43 min., 70 eV): 170 (3, [M]+⋅), 123 (39), 95 (18), 83 (19), 81 (41), 71 (18), 70 (18), 69 (21), 67 (26), 55 (100), 41 (45).

### b) Stereoisomer Separation

3,7-Dimethylnon-6-en-1-ol (IV) has been transformed to the corresponding 3,7-dimethylnon-6-en-1-yl 4-nitrobenzoate (VII) to enable for separation into individual isomers by SFC (supercritical fluid chromatography), followed by ester hydrolysis to the individual compounds (R,E)-(IV), (S,E)-(IV), (R,Z)-(IV), and (S,Z)-(IV).
i) 3,7-dimethylnon-6-en-1-yl 4-nitrobenzoate (VI):

To a stirred solution of compound IV (5 g, 29 mmol, 1 equiv) in DCM (100 mL) at 0° C. were added p-nitrobenzoic acid (2) (7.35 g, 44 mmol, 1.5 equiv), DCC (11.3 g, 55 mmol, 1.9 equiv), and DMAP (1.41 g, 11.6 mmol, 0.4 equiv). It was stirred at rt for 16 h. The reaction mixture was filtered, and the filtered solid was washed with DCM (50 mL). The filtrate was washed with water (200 mL), dried over sodium sulfate and concentrated under reduced pressure. The crude product (VII) was purified by HPLC to afford 3 (6 g, 64%) as a colourless liquid.
ii) SFC separation:

Compound VII (6 g) was subjected to SFC purification (15% co-solvent: 0.5% isopropyl amine in methanol, Column: Lux A1 (250×30 mm, 5 μ), Flow rate: 3 ml/min) to obtain four fractions (S,E)-(VIIc), (S,Z)-(VIIa), (R,E)-(VIIb) and (R,Z)-(VIId).
iii) Ester hydrolysis:

To a stirred solution of compound (S,Z)-(VII) in THF-MeOH-H₂O (7:2:1 vol) at rt was added LiOH. H₂O (2 equiv). It was stirred at rt for 2 h. The reaction mixture was concentrated under reduced pressure, diluted with water and extracted with EtOAc. The combined organic layer was dried over sodium sulfate and concentrated under reduced pressure. The crude product was purified by flash column chromatography using 6-7% EtOAc in pet ether as eluent to afford (S,Z)-(V) ((S,Z)-3,7-dimethylnon-6-en-1-ol) as a colorless liquid.

Compounds (R,E)-(VII), (S,E)-(VII) and (R,Z)-(VII) have been hydrolysed accordingly to yield compounds (R,E)-(V) ((R,E)-3,7-dimethylnon-6-en-1-ol), (S,E)-(V) ((S,E)-3,7-dimethylnon-6-en-1-ol) and (R,Z)-(V) ((R,Z)-3,7-dimethylnon-6-en-1-ol), respectively. The configuration of the individual isomers has been assigned by chiral GC and NMR analysis.

### Example 2: Synthesis of All Four Isomers of 3,7-dimethylnon-6-enal

General procedure: To a solution of Dess-Martin periodinane (1.2 equiv) in CH₂Cl₂ (0.02-0.09 M) was added dropwise at 0° C. a solution of the alcohol V (1.0 equiv) in CH₂Cl₂ (0.02-0.12 M). The reaction mixture was stirred for 3 hours at room temperature, quenched with 2M NaOH. The layers were separated and the aqueous layer was extracted with CH₂Cl₂. The combined organic layers were washed with sat. Na₂S₂O₃, sat. brine, dried over MgSO4 and filtered. The solvent was removed under reduced pressure and the crude was purified by column chromatography (SiO₂, Biotage or Pasteur pipette, heptane/MTBE mixtures).

### a) (R,E)-3,7-dimethylnon-6-enal ((R,E)-Ia) Obtained From (R,E)-V

(R,E)-3,7-dimethylnon-6-en-1-ol (50 mg) was oxidized as described in the general procedure to give (R,E)-3,7-dimethylnon-6-enal (10 mg, 20% yield) as a colorless oil.

¹H NMR (600 MHz, C₆D₆) δ (ppm)=9.35 (t, J=1.5 Hz, 1H), 5.11 (t, J=7.0 Hz, 1H), 2.02-1.67 (m, 7H), 1.54 (s, 3H), 1.22-1.13 (m, 1H), 1.09-1.03 (m, 1H), 0.99 (t, J=7.5 Hz, 3H), 0.74 (d, J=6.4 Hz, 3H). ¹³C NMR (151 MHZ, C₆D₆) δ (ppm)=201.2, 137.3, 123.5, 51.3, 37.5, 33.1, 28.1, 26.0, 20.2, 16.3, 13.4. GC-MS (El) m/z (%): 168 (1, [M]^{+⋅}), 121 (25), 95 (53), 83 (27), 69 (31), 67 (22), 55 (100), 43 (22), 41 (65), 39 (27), 29 (45).

Chiral GC (Sigma-Aldrich, Astec Chiraldex G-DP, 2 min@50° C.-1° C./min-100° C.-5° C./min-2 min@200° C.): t_{R}/min=56.67 min.

Odor description: aldehydic, waxy, fatty, floral, rosy, citrus, juicy, citronella, watery, precyclemone, myraldene, green.

GCTH: 0.013 ng.

### b) (S,E)-3,7-dimethylnon-6-enal ((S,E)-la) Obtained From (S,E)-V)

(S,E)-3,7-dimethylnon-6-en-1-ol (0.20 g) was oxidized as described in the general procedure to give (S,E)-3,7-dimethylnon-6-enal (89 mg, 45% yield) as a colorless oil.

¹H NMR and ¹³C NMR data are identical to example 2a. GC-MS (El) m/z (%): 168 (1, [M]^{+⋅}), 121 (22), 95 (45), 83 (29), 69 (29), 67 (21), 55 (100), 41 (64), 39 (28), 29 (46), 27 (22). Chiral GC (Sigma-Aldrich, Astec Chiraldex G-DP, 2 min@50° C.-1° C./min-100° C.-5° C./min-2 min@200° C.): t_{R}/min=56.45 min.

Odor description: aldehydic, waxy, fatty, green, citrus leaf, citrus, citrus hystrix, citronellal, verbena, floral, rosy.

GCTH: 1.4 ng.

### c) (S,Z)-3,7-dimethylnon-6-enal ((S,Z)-Ia) Obtained From (S,Z)-V

(S,Z)-3,7-dimethylnon-6-en-1-ol (25 mg) was oxidized as described in the general procedure to give (S,Z)-3,7-dimethylnon-6-enal (10 mg, 43% yield) as a colorless oil. ¹H NMR (600 MHZ, C₆D₆) δ (ppm)=9.35 (t, J=2.0 Hz, 1H), 5.06 (t, J=7.2 Hz, 1H), 2.00-1.75 (m, 6H), 1.75-1.69 (m, 1H), 1.66 (d, J=1.1 Hz, 3H), 1.19-1.11 (m, 1H), 1.07-0.97 (m, 1H), 0.93 (t, J=7.7 Hz, 3H), 0.73 (d, J=6.8 Hz, 3H). ¹³C NMR (151 MHZ, C₆D₆) δ (ppm)=201.2, 137.5, 124.7, 25 51.3, 37.8, 28.1, 25.7, 25.4, 23.4, 20.1, 13.4. GC-MS (El) m/z (%): 168 (1, [M]+⋅), 121 (24), 95 (50), 83 (27), 69 (29), 67 (19), 55 (100), 41 (66), 39 (27), 29 (44), 27 (22).

Chiral GC (Sigma-Aldrich, Astec Chiraldex G-DP, 2 min@50° C.-1° C/min-100° C.-5° C./min-2 min@200° C.): t_{R}/min=55.38 min.

Odor description: aldehydic, waxy, fatty, green, fatty, metallic, citrus leaf, citrus, juicy, citronellal.

GCTH: 0.75 ng.

### d) (R,Z)-3,7-dimethylnon-6-enal ((R,Z)-Ia) Obtained From (R,Z)-V

(R,Z)-3,7-dimethylnon-6-en-1-ol (0.20 g) was oxidized as described in the general procedure to give (R,Z)-3,7-dimethylnon-6-enal (0.10 g, 56% yield) as a colorless oil.

¹H NMR and ¹³C NMR data are identical to example 2c. GC-MS (El) m/z (%): 168 (1, [M]+⋅), 121 (28), 95 (50), 83 (27), 69 (30), 67 (21), 55 (100), 41 (66), 39 (28), 29 (46), 27 (21).

Chiral GC (Sigma-Aldrich, Astec Chiraldex G-DP, 2 min@50° C.-1° C/min-100° C.-5° C./min-2 min@200° C.): t_{R}/min=55.54 min.

Odor description: aldehydic, waxy, fatty, green, metallic, fatty, citrus leaf, citrus, juicy, fruity, citronellal.

GCTH: 1.2 ng.

### Example 3: Synthesis of an Isomeric Aldehyde Mixture Ia, Ib, Ic, II:

An autoclave was charged with 3,7-dimethylnona-2,6-dienal (8.0 g, 48.1 mmol, 1.0 equiv, prepared as described in WO2014140032 A1), 5% wet Pd/C (0.20 g, 0.094 mmol, 0.2 mol %, 58% water content) and Na₂CO₃ (0.10 g, 0.94 mmol, 2 mol %). The reaction mixture was then hydrogenated at room temperature for 22 hours at 3 bar H₂. The crude was purified by Kugelrohr distillation to give a mixture of aldehydes (6.1 g, 75% yield, 53% of (E)-3,7-dimethylnon-6-enal (E-Ia)+29% of (Z)-3,7-dimethylnon-6-enal (Z-Ia)+13% of 3,7-dimethylnon-7-enal (Ib, sum of E/Z isomers)+1% of 3-methyl-7-methylenenonanal (Ic)+4% of 3,7-dimethylnonanal (II)) as a colorless oil.

Characteristic ¹³C NMR data for (E)-Ib (olefinic signals): ¹³C NMR (101 MHZ, CDCl₃) δ (ppm)=135.5 (C_{q}), 118.3 (CH).

Characteristic ¹³C NMR data for (Z)-Ib (olefinic signals): ¹³C NMR (101 MHZ, CDCl₃) δ (ppm)=135.7 (C_{q}), 119.0 (CH).

Characteristic ¹³C NMR data for Ic (olefinic signal): ¹³C NMR (101 MHZ, CDCl₃) δ (ppm)=107.6 (═CH₂).

A reference sample of 3,7-dimethylnonanal (II) was prepared according to a literature procedure FR2124279 A1. The analytical data for the mixture of two diastereoisomers were as follows: ¹³C NMR (101 MHZ, CDCl₃) δ (ppm)=203.1, 51.1, 51.1, 36.9, 34.3, 29.5, 29.4, 28.2, 28.2, 24.4, 24.4, 20.0, 19.9, 19.2, 19.1, 11.4, 11.4.

Odor description: fresh citrus metallic (orange, juicy) green

GCTH: 0.19 ng.

### Example 4: Synthesis of 2,3,7-trimethylnon-6-en-1-ol IIIa

### a) 3,7-dimethyl-2-methylenenon-6-enal

3,7-dimethylnon-6-enal (2.50 g, 14.9 mmol, 1.0 equiv, prepared according to example 2 from a racemic E/Z mixture of 3,7-dimethylnon-6-en-1-ol) was alpha-methylenated using a literature procedure (WO2018024820A1, example 1) to give after Kugelrohr distillation 3,7-dimethyl-2-methylenenon-6-enal (1.92 g, 71% yield, mixture of E/Z isomers 57:43).

¹³C NMR (101 MHZ, CDCl₃) δ (ppm)=194.6, 155.4, 155.4, 137.4, 137.1, 133.0, 123.7, 122.5, 35.9, 35.6, 32.3, 31.0, 31.0, 25.6, 25.3, 24.7, 22.8, 19.5, 19.5, 15.8, 12.8, 12.7.

Odor description: fatty, aldehydic, metallic, fishy, citrus citral.

### b) 2,3,7-trimethylnon-6-enal

3,7-dimethyl-2-methylenenon-6-enal (1.80 g, 9.98 mmol, 1.0 equiv) was hydrogenated using a literature procedure (WO2018024820A1, example 2) to give after Kugelrohr distillation 2,3,7-trimethylnon-6-enal (1.50 g, 78% yield, mixture of 4 isomers) as a colorless oil.

¹³C NMR (101 MHZ, CDCl₃) δ (ppm)=205.7, 205.7, 205.6, 205.6, 137.5, 137.3, 137.3, 123.6, 123.5, 122.4, 122.3, 51.5, 51.5, 50.5, 35.0, 34.7, 33.4, 32.3, 32.3, 32.1, 32.1, 25.4, 24.7, 22.8, 22.8, 17.3, 17.3, 15.9, 15.4, 15.3, 12.8, 12.7, 9.8, 9.8, 8.1, 8.1

Odor description: citrus citral, lemongrass, fatty, aldehydic.

GCTH: 16 ng.

### c) 2,3,7-trimethylnon-6-en-1-ol

To a solution of NaBH₄ (0.10 g, 2.63 mmol, 0.6 equiv) in ethanol (4 mL) was added at room temperature a solution of 2,3,7-trimethylnon-6-enal (0.80 g, 4.39 mmol, 1.0 equiv) in ethanol (4 mL). After stirring for 3 hours at that temperature, the reaction mixture was quenched with water and diluted with MTBE. The layers were separated and the organic layer was washed with sat. NaHCO₃ and brine, dried over MgSO₄ and concentrated under reduced pressure. The crude was purified by column chromatography and Kugelrohr distillation to afford 2,3,7-trimethylnon-6-en-1-ol IIIa (0.48 g, 59% yield, mixture of 4 isomers in a 15:24:25:36 ratio) as a colorless oil.

¹H NMR (400 MHZ, CDCl₃) δ (ppm)=5.26-4.95 (m, 1H), 3.72-3.50 (m, 1H), 3.49-3.39 (m, 1H), 2.13-1.85 (m, 4H), 1.74-1.45 (m, 6H), 1.43-1.30 (m, 1H), 1.26-1.06 (m, 1H), 0.97(m, 3H), 0.92-0.87 (m, 3H), 0.81 (m, 3H). ¹³C NMR (101 MHZ, CDCl₃) δ (ppm)=136.9, 124.4, 124.3, 123.2, 123.1, 66.8, 66.0, 66.0, 40.6, 39.5, 35.4, 35.1, 34.1, 33.0, 32.3, 25.7, 24.7, 22.8, 16.9, 16.8, 15.8, 15.8, 14.3, 13.5, 13.5, 12.8, 11.3, 12.8.

Odor description: fatty rosy, citronellol, slightly waxy dusty.

GCTH: 0.04 ng.

### Example 5: Synthesis of (2-methyl-2-(4-methylhex-3-en-1-yl)cyclopropyl)methanol IIIb

A solution of diethylzinc (69.5 mL, 69.5 mmol, 1 M in hexane, 1.3 equiv) in CH₂Cl₂ (60 mL) was added at −15° C. diiodomethane (10.79 mL, 134 mmol, 2.5 equiv). The reaction mixture was then allowed to stir at the same temperature for 1 hour. To this was added a solution of 3,7-dimethylnona-2,6-dien-1-ol (9 g, 53.5 mmol, 1.0 equiv, prepared according to Rojahn, W. et al. Dragoco Report (German Edition) 1978, 25, 248) in CH₂Cl₂ (50 mL) at −15° C. The reaction mixture was then allowed to stir at room temperature for 20 h. The reaction mixture was quenched by adding aqueous ammonium chloride solution (50 mL) at 15° C., diluted with water (50 mL) and filtered through celite bed. Filtrate was extracted with CH₂Cl₂ (2×250 mL). The combined organic layer was dried over anhydrous sodium sulphate, filtered and concentrated under reduced pressure to afford crude as pale yellow liquid was purified twice using AgNO₃ impregnated 230-400 size silica gel eluting with 4-5% ethyl acetate in petroleum ether to afford (2-methyl-2-(4-methylhex-3-en-1-yl)cyclopropyl)methanol IIIb (4.7 g, 48% yield, mixture of 4 isomers in a 8:25:15:52 ratio) as colorless oil.

¹H NMR (400 MHZ, CDCl₃) δ (ppm)=5.18-5.02 (m, 1H), 3.78-3.42 (m, 2H), 2.16-1.92 (m, 4H), 1.71-1.59 (m, 3H), 1.44-1.30 (m, 2H), 1.22-1.11 (m, 1H), 1.11-1.06 (m, 3H), 1.01-0.84 (m, 4H), 0.56-0.43 (m, 1H), 0.17-0.07 (m, 1H). ¹³C NMR (101 MHZ, CDCl₃) δ (ppm)=137.3, 137.0, 136.9, 136.7, 124.2, 122.9, 122.9, 63.9, 63.5, 41.4, 41.1, 34.5, 34.2, 32.3, 27.3, 26.2, 25.6, 25.3, 25.3, 25.0, 24.7, 24.3, 24.3, 22.8, 20.2, 20.2, 19.9, 17.7, 17.6, 17.6, 17.5, 17.0, 17.0, 15.8, 12.8, 12.7.

Odor description: floral, rosy, ethyl citronellol, fruity litchy.

GCTH: 1.2 ng.

### Example 6: Synthesis of 3.7-dimethylnonan-1-ol IIIc

An autoclave was charged with 3,7-dimethylnona-2,6-dienal (2.0 g, 12.0 mmol, 1.0 equiv, prepared as described in WO2014140032 A1), Raney-Ni (0.65 g, washed with ethanol before use) and 20 mL ethanol. The reaction mixture was hydrogenated at 30 bar and 100° C. for 2 hours. After filtration and concentration, the crude was purified by chromatography to give 3,7-dimethylnonan-1-ol IIIc (0.97 g, 47% yield, 2 diastereoisomers) as a colorless oil.

¹H NMR (400 MHZ, CDCl₃) δ (ppm)=3.77-3.59 (m, 2H), 1.67-1.50 (m, 2H), 1.44-0.97 (m, 11H), 0.94-0.80 (m, 9H). ¹³C NMR (101 MHZ, CDCl₃) δ (ppm)=61.2, 40.0, 40.0, 37.5, 37.4, 36.9, 36.8, 34.4, 29.5, 29.5, 29.5, 29.4, 24.4, 19.4, 11.4, 11.4.

Odor description: floral, rosy, peony, fruity, litchi, grape, green, waxy, hot iron.

GCTH: 3.1 ng.

### Example 7: Synthesis of 4,8-dimethyldec-7-en-2-ol IIId

### a) 3,7-dimethylnon-6-enoic acid

To a solution of 3,7-dimethylnon-6-en-1-ol (23 g, 135 mmol) in acetone (230 mL) at 0° C. was added Jones' reagent (135 mL) over a period of 1 h. The reaction mixture was stirred at 25° C. for 1 h. Acetone was concentrated under reduced pressure, diluted with water (350 mL) and extracted with ethylacetate (1×700 mL). The organic extract was washed with 5% NaOH solution (1×300 mL). The aqueous layer was acidified to pH=˜1-2 using conc. HCl (55 mL). The product was extracted with ethylacetate (2×600 mL). The combined organic layer was dried over anhydrous sodium sulphate, filtered, and concentrated under reduced pressure. The crude product was purified by chromatography using 8% ethylacetate-petroleum ether to afford 3,7-dimethylnon-6-enoic acid (8 g, 32%) as a colorless oil.

### b) N-methoxy-N,3,7-trimethylnon-6-enamide

To a suspension of 3,7-dimethylnon-6-enoic acid (15 g, 81 mmol) and N,O-dimethylhydroxylamine hydrochloride (10.3 g, 106 mmol) in CH₂Cl₂ (150 mL) were added EDC*HCl (20.29 g, 106 mmol) and DMAP (15.91 g, 130 mmol) at 25° C. The mixture was stirred at room temperature for 16 h. The reaction mixture was diluted with water (300 mL) and extracted with CH₂Cl₂ (2×250 mL). The combined organic layer was washed with 1.5 N HCl solution (1×150 mL), brine solution (1×150 mL), dried over Na₂SO₄ and concentrated under reduced pressure. The crude product was purified by chromatography using 8-10% ethylacetate and petroleum ether as an eluent to afford N-methoxy-N,3,7-trimethylnon-6-enamide (15.6 g, 84%) as colorless liquid.

### c) 4,8-dimethyldec-7-en-2-one

To a stirred solution of N-methoxy-N, 3,7-trimethylnon-6-enamide (17 g, 74.8 mmol) in THF (160 mL) was added methyl magnesium bromide (3.0 M in diethyl ether) (40 mL, 120 mmol) dropwise at 0° C. under inert atmosphere. After the addition, the reaction mixture was allowed to stir at 25° C. for 1 h. After completion of the reaction, the reaction mass was quenched with saturated aq. NH₄Cl solution (150 mL) at 5° C., extracted with ethyl acetate (2×250 mL). The combined organic layer was washed with brine solution (1×100 mL), dried over sodium sulfate and concentrated under reduced pressure. The crude product was purified by chromatography using 4-6% ethyl acetate in petroleum ether as eluent to afford 4,8-dimethyldec-7-en-2-one (11.5 g, 84%) as colorless liquid.

Odor description: fatty(soapy) fruity citrus.

### d) 4,8-dimethyldec-7-en-2-ol

To a stirred solution of 4,8-dimethyldec-7-en-2-one (6.4 g, 35.1 mmol) in methanol (65 mL) at 0° C. was added NaBH₄ (0.664 g, 17.5 mmol) in portion. The reaction mixture was stirred at room temperature for 1.5 h. The reaction mixture was cooled to 0° C. and quenched with ice water (40 mL) and extracted with ethyl acetate (2×150 mL). The combined organic layer was washed with brine (1×60 mL), dried over anhydrous sodium sulfate, filtered, and concentrated under reduced pressure. The crude product was purified by chromatography using 7 to 8% ethylacetate & petroleum ether as an eluent to obtain 4,8-dimethyldec-7-en-2-ol (5.25 g, 81%, mixture of 4 isomers in a 8:28:60:4 ratio) as colorless oil.

¹³C NMR (101 MHZ, CDCl₃, 2 major isomers) δ (ppm)=124.3, 124.3, 123.1, 66.2, 65.8, 46.9, 46.8, 38.0, 37.7, 37.3, 37.0, 32.3, 29.5, 29.5, 29.1, 25.3, 25.2, 25.0, 24.7, 24.3, 24.3, 23.6, 23.6, 22.9, 20.1, 19.3, 19.3, 15.8, 12.8, 12.8.

Odor description: floral, rosy, peony, pomelol, green, grapefruit, waxy, fatty, slightly stearate.

GCTH: 15 ng.

### Example 8: Biodegradation Results for the Compound Ia (mixture of four stereoisomers)

Manometric Respirometry test (OECD guideline for the testing of materials No. 301F, Paris 1992) was used to access biodegradability of
a) the compound Ia (mixture of four stereoisomers), and
b) the mixture of example 3 (aldehyde mixture Ia, Ib, Ic, II).

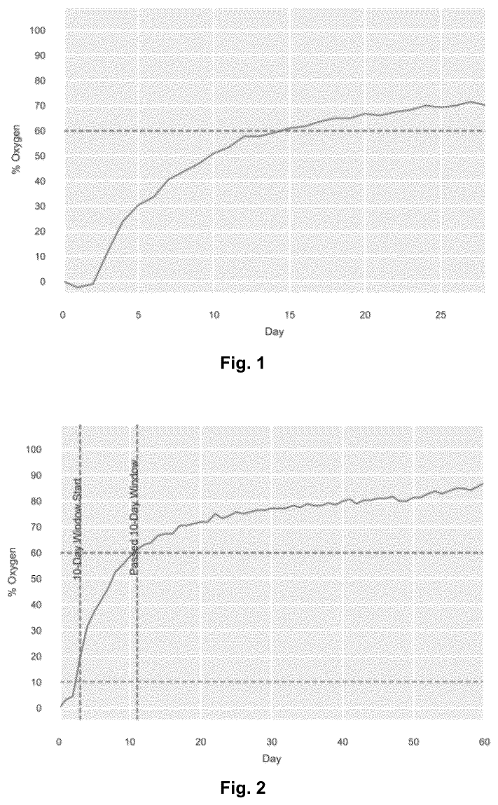

The results show that
a) the compound Ia (mixture of four stereoisomers) shows a BOD (biological oxygen demand) of 70% (60 days, readily biodegradable) (FIG. 1), and
b) the mixture of example 3 (aldehyde mixture Ia, Ib, Ic, II) shows a BOD (biological oxygen demand) of 87% (60 days, readily biodegradable) (FIG. 2).

A compound can be classified biodegradable, if it reaches the pass level of 60% oxygen consumption of theory required for complete mineralization.

It is readily biodegradable, if the pass level is reached within 10 days within the 28-day period of the test. The 10-day window begins when the degree of biodegradation has reached 10%.

If the pass level is obtained after 28-day period of the test, the compound can be classified as inherently biodegradable.

### Example 9: Perfumery Example

The basic accord REF has been modified by addition of different compounds according to the invention and comparative compounds as outlined in Table 1.

TABLE 1

| INGREDIENT NAME | REF | A | B | C | D | E | F |
|---|---|---|---|---|---|---|---|
| ALDEHYDE C 10 DECYLIC | 3 | 3 | 3 | 3 | 3 | 3 | 3 |
| BERGAMOT REC ABX5638 | 90 | 90 | 90 | 90 | 90 | 90 | 90 |
| CASSYRANE | 10 | 10 | 10 | 10 | 10 | 10 | 10 |
| CITRONELLOL EXTRA | 80 | 80 | 80 | 80 | 80 | 80 | 80 |
| CORPS PAMPLEMOUSSE PURE | 3 | 3 | 3 | 3 | 3 | 3 | 3 |
| 10%/TEC | | | | | | | |
| DIPROPYLENE GLYCOL | 4.5 | 4.5 | 4.5 | 4.5 | 4.5 | 4.5 | 4.5 |
| ETHYL MALTOL 1%/BA | 3 | 3 | 3 | 3 | 3 | 3 | 3 |
| FLOROPAL | 16 | 16 | 16 | 16 | 16 | 16 | 16 |
| GALAXOLIDE S | 40 | 40 | 40 | 40 | 40 | 40 | 40 |
| GARDENOL | 8 | 8 | 8 | 8 | 8 | 8 | 8 |
| GERANYL ACETATE SYNTHETIC | 60 | 60 | 60 | 60 | 60 | 60 | 60 |
| HEDIONE | 80 | 80 | 80 | 80 | 80 | 80 | 80 |
| HEXENYL-3-CIS ACETATE | 10 | 10 | 10 | 10 | 10 | 10 | 10 |
| HEXYL ACETATE | 80 | 80 | 80 | 80 | 80 | 80 | 80 |
| HEXYL SALICYLATE | 80 | 80 | 80 | 80 | 80 | 80 | 80 |
| ISORALDEINE 70 | 30 | 30 | 30 | 30 | 30 | 30 | 30 |
| JAVANOL | 1 | 1 | 1 | 1 | 1 | 1 | 1 |
| LEMON TERPENES CITRAL FREE | 66 | 66 | 66 | 66 | 66 | 66 | 66 |
| LINALOOL SYNTHETIC | 120 | 120 | 120 | 120 | 120 | 120 | 120 |
| MANDARINAL 32048 SAE/2 | 20 | 20 | 20 | 20 | 20 | 20 | 20 |
| MANZANATE | 10 | 10 | 10 | 10 | 10 | 10 | 10 |
| ORANGE OIL PHASE DIST | 80 | 80 | 80 | 80 | 80 | 80 | 80 |
| PETALIA | 30 | 30 | 30 | 30 | 30 | 30 | 30 |
| POMAROSE | 1 | 1 | 1 | 1 | 1 | 1 | 1 |
| RASPBERRY KETONE | 1 | 1 | 1 | 1 | 1 | 1 | 1 |
| ROSE OXIDE CO | 0.5 | 0.5 | 0.5 | 0.5 | 0.5 | 0.5 | 0.5 |
| TRICYCLAL | 3 | 3 | 3 | 3 | 3 | 3 | 3 |
| mixture of four isomers Ia | | 3 | | | | | 3 |
| citronellal | | | 10 | 30 | | | |
| aldehyde mixture Ia, Ib, Ic, II | | | | | 3 | | |
| 2,3,7-trimethylnon-6-en-1-ol IIIa | | | | | | 60 | 60 |
| DPG | 70 | 67 | 60 | 40 | 67 | 10 | 7 |
| total | 1000 | 1000 | 1000 | 1000 | 1000 | 1000 | 1000 |

REF: This accord presents a fresh citrus grapefruit top note character, with a floral rosy musky base note. It is intended for application in shampoo or shower gel. The present application is in shampoo at 0.8%.
A: The addition of 0.3% of a mixture of four isomers of Ia brings already a fresh, aldehydic, vibrant facet as well as a citrus juicy facet, which reinforces the natural character, as well as the perceived efficacy olfactive cue.
B: By comparison, addition of 1% citronellal do bring also some juiciness, but tends to give a citronella olfactive cue, which is usually not consumer liked, especially in Europe and USA.
C: Addition of 3% citronellal gives a clear citronella character, even more polarizing for consumer preference.
D: Addition of 0.3% of the isomeric aldehyde mixture of example 3 brings a green, vibrant, metallic and citrus lemongrass effect, which also reinforces naturality and perceived efficacy, with a clearly different olfactive contribution than a mixture of four isomers of Ia.
E: Addition of 6% compound of example 4 (IIIa) brings a soft floral rosy depth to the accord, which reinforces the juicy citrus olfactive aspect and the caring naturality hedonic aspect.
F: Combination of 0.3% of mixture of four isomers of Ia and 6% of compound of example 4 (IIIa) combines both the vibrant naturality for the lift and top note, and the caring juicy aspect for the hedonic and bloom in water.

1. A compound of formula (R,E)-(Ia), which is (R,E)-3,7-dimethylnon-6-enal

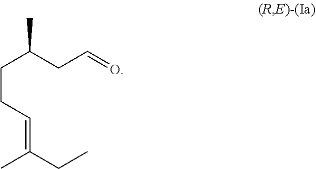

2. An isomeric mixture comprising the compound of formula (R,E)-(Ia) according to claim 1 and a compound selected from the group consisting of the compound of formula (S,E)-(la), (R,Z)-(Ia), (S,Z)-(Ia) and combinations thereof:

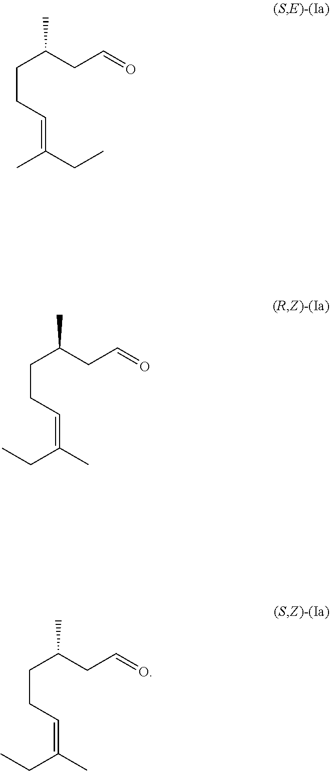

3. (canceled)

4. A fragrance composition comprising the compound of formula (R,E)-(Ia) according to claim 1.

5. The fragrance composition according to claim 4, further comprising a compound selected from the group consisting of a compound of formula (Ib), (Ic), (II) and combinations thereof: 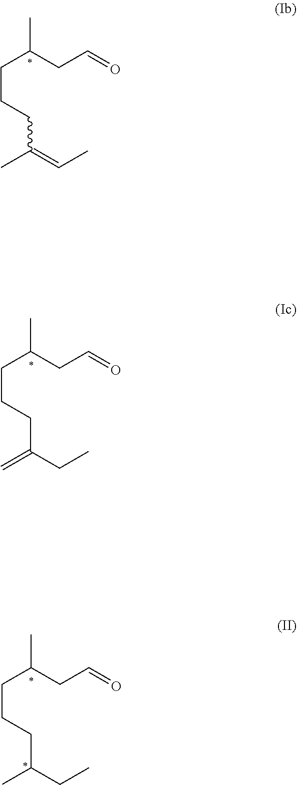
wherein the wavy bond is indicates an unspecified configuration of the adjacent double bond, and the star is indicates a stereocenter at the C-atom.

6. The fragrance composition according to claim 4 or claim 5, further comprising at least one compound of formula (III) 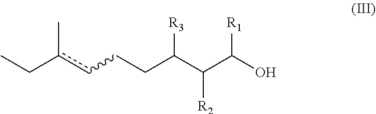
wherein [symbol]
indicates a carbon-carbon single or double bond,
the wavy bond is indicates an unspecified configuration of the adjacent double bond, if present,
and R₁, R₂ and R₃ are independently selected from H or Me,
or wherein R₁ is H or Me, and R₂ and R₃ form together with the carbon atoms they are attached to a cyclopropyl ring.

7. (canceled)

8. A compound of formula (III) 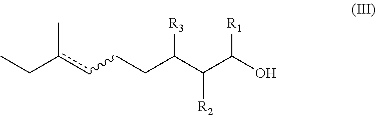
wherein [symbol]
indicates a carbon-carbon single or double bond,
and the wavy bond indicates an unspecified configuration of the adjacent double bond, if present
and R₁, R₂ and R₃ are independently selected from H or Me,
or wherein R₁ is H or Me, and R₂ and R₃ form together with the carbon atoms they are attached to a cyclopropyl ring,
with the proviso that the compound is not 3,7-dimethylnonan-1-ol.

9. (canceled)

10. A fragrance composition comprising at least one compound of formula (III) according to claim 8.

11. The fragrance composition according to claim 4, further comprising at least one compound of formula (X) 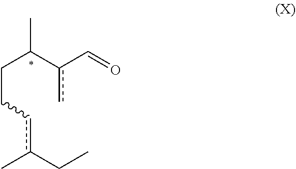
wherein [symbol]
indicates a carbon-carbon single or double bond,
the wavy bond indicates an unspecified configuration of the adjacent double bond, if present, and the star indicates a stereocenter at the C-atom.

12. A consumer product comprising the compound of formula (R,E)-(Ia) according to claim 1, and a consumer product base.

13. A consumer product comprising the compound of formula (III) according to claim 8, and a consumer product base.

14. The consumer product according to claim 12, wherein the consumer product base is selected from fine fragrance, household products, laundry products, body care products, cosmetic products and air care products.

15. A method of improving, conferring, enhancing or modifying a consumer product base by adding thereto an olfactory acceptable amount of the compound of formula (R,E)-(Ia) according to claim 1.

16. A fragrance composition comprising the isomeric mixture according to claim 2.

17. The fragrance composition according to claim 5, further comprising at least one compound of formula (III) 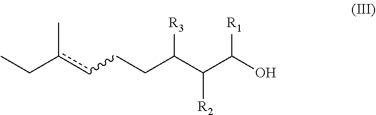
wherein [symbol]
indicates a carbon-carbon single or double bond,
the wavy bond indicates an unspecified configuration of the adjacent double bond, if present,
and R₁, R₂ and R₃ are independently selected from H or Me,
or wherein R₁ is H or Me, and R₂ and R₃ form together with the carbon atoms they are attached to a cyclopropyl ring.

18. A consumer product comprising the isomeric mixture according to claim 2, and a consumer product base.

19. A consumer product comprising the fragrance composition according to claim 4, and a consumer product base.

20. A consumer product comprising the fragrance composition according to claim 6, and a consumer product base.

21. A consumer product comprising the fragrance composition according to claim 11, and a consumer product base.

22. A consumer product comprising the fragrance composition according to claim 10, and a consumer product base.

23. The consumer product according to claim 13, wherein the consumer product base is selected from fine fragrance, household products, laundry products, body care products, cosmetic products and air care products.

24. A method of improving, conferring, enhancing or modifying a consumer product base by adding thereto an olfactory acceptable amount of the isomeric mixture according to claim 2.

25. A method of improving, conferring, enhancing or modifying a consumer product base by adding thereto an olfactory acceptable amount of the fragrance composition according to claim 4.

26. A method of improving, conferring, enhancing or modifying a consumer product base by adding thereto an olfactory acceptable amount of the fragrance composition according to claim 6.

27. A method of improving, conferring, enhancing or modifying a consumer product base by adding thereto an olfactory acceptable amount of the fragrance composition according to claim 11.

28. A method of improving, conferring, enhancing or modifying a consumer product base by adding thereto an olfactory acceptable amount of the fragrance composition according to claim 8.
